(19) United States
(12) Patent Application Publication
(10) Pub. No.: US 2011/0098633 A1
(43) Pub. Date: Apr. 28, 2011

(54) ULTRASONIC TIP FOR SINUS MEMBRANE ELEVATION

(76) Inventors: Andreas KURREK, Ratingen (DE); Marcel A. WAINWRIGHT, Dusseldorf (DE); Angelo TROEDHAN, Wien (AT); Francis DIERAS, Bordeaux (FR)

(21) Appl. No.: 12/981,674

(22) Filed: Dec. 30, 2010

Related U.S. Application Data
(62) Division of application No. 11/822,760, filed on Jul. 10, 2007.

(30) Foreign Application Priority Data
May 4, 2007 (FR) 0754881

(51) Int. Cl. A61B 17/16

(52) U.S. Cl. 604/22

(57) ABSTRACT

Method for lifting a sinus membrane located between a sinus and a maxilla that involves the drilling of a hole of a given diameter in the maxilla reaching the sinus membrane, and includes introducing in the hole an ultrasonic tip having a distal part including a ring-shaped part of increasing cross-section and a distal end formed by a flat area extending generally perpendicular to the longitudinal axis of the distal part. The flat area of the distal part has a distal side facing away from the proximal part and is bordered by a rounding, the ring shaped part being larger than the body of the tip but closely corresponding to the diameter of the hole. The tip includes an internal irrigation channel emerging on the distal side of the flat area. While the tip is within the hole, the tip is advanced towards the membrane while ultrasound vibrations are fed to the tip and irrigation fluid is supplied to the internal irrigation channel. This creates a cavitation effect in the irrigation fluid between the tip and the membrane and a lifting force that gently detaches and elevates the sinus membrane without requiring a direct contact between the membrane and the distal part of the tip.

### RELATED APPLICATION DATA

This application is a divisional of application Ser. No. 11/822,760 filed Jul. 10, 2007, the entirety of which is incorporated herein by reference.

### BACKGROUND TO THE INVENTION

The present invention concerns the field of dental implants and in particular devices used to prepare the implantation site in odontology.

For long-term integration in the maxillary bone (osteointegration), the implant must be fixed in a sufficient volume of cortical bone. However, the maxilla does not always have a sufficient thickness or volume of cortical bone for the stable placement of dental implants. In this case, the bone mass must be augmented on the sinus side of the maxilla. The bone graft may come from the patient himself (autogenous bone graft) or from an external source (artificial bone graft).

FIGS. 1A to 1D show the various stages implemented during a maxillary bone augmentation procedure on a patient's upper jaw. FIG. 1A represents a maxilla 1 delimiting a cavity corresponding to a sinus 2. A dental implant must be fixed in the lower part of the maxilla 1. At this site of the maxilla 1 the thickness e of cortical bone is insufficient for placement of the implant so that bone augmentation on the sinus side 2 is required. After cutting open the gum (not shown), the physician first drills a hole in the maxilla emerging in sinus 2 (FIG. 1B). The maxilla 1 is separated from the sinus 2 by the sinus floor which is composed of a sinus membrane 3, also called Schneider's membrane, in contact with the inner wall of maxilla 1. Once the maxilla is pierced, the sinus membrane 3 must be lifted in order to free a volume for the insertion of the grafted bone substance. The instruments used for this purpose make it possible to lift the sinus membrane.

As shown on FIG. 1C, the sinus membrane 3 may be lifted using a balloon 4 introduced into sinus 2 by means of an instrument 5 via the hole drilled in the maxilla 1. Inflation of the balloon 4 pushes back the sinus membrane and frees a volume between the latter and the cortical bone of the maxilla 1. This volume is then filled with a bone graft 6 as shown on FIG. 1D.

The sinus membrane may also be elevated with other types of blunt instruments.

Elevation of the sinus membrane is a very delicate operation as it is very fragile and may be torn at any time by the instrument used in direct contact with it. Such a tearing seriously compromises the success of implantation in particular because of the dispersion of the bone substance in the sinus and the incurred risks of infection.

Instruments currently used to lift the membrane, even those using a balloon, are in contact with only a very small area of the membrane so that the latter may be easily torn when the physician applies too strong a pressure with the instrument. It is all the more difficult for the physician to control the pressure exerted on the membrane because of the poor visibility of the site and the depth of the approach. Moreover, certain instruments may be too invasive.

### OBJECT AND SUMMARY OF THE INVENTION

The purpose of the present invention is to propose a new instrument designed to lift the sinus membrane without tearing it.

This goal is achieved by using an ultrasonic tip consisting of a body extending between a proximal part adapted for mechanical coupling to a surgical handpiece generating ultrasound vibrations and a distal part intended to reproduce the ultrasound vibrations transmitted by the handpiece. This distal part comprises a ring-shaped part of increasing cross section with an end formed by a flat surface more or less perpendicular to the longitudinal axis the distal part. The body further presents a bent or curved shape between the proximal part and the distal part. The tip also comprises an internal irrigation channel ending in the aforesaid flat surface.

The ultrasonic tip of the present invention therefore has a design that permits the safe lifting of the sinus membrane. The distal part of the instrument which is intended to be introduced under the sinus membrane and lift it, has a flat surface so that even in the case of contact between the distal part of the tip and the membrane, the risks of tearing the latter are considerably reduced in comparison with the instruments conventionally used which have a blunt or similar shape at their site of contact with the membrane.

Moreover, according to the invention, the tip has an internal irrigation channel emerging on the flat surface of the distal part. Thus, when the tip of the invention is introduced into the drilled hole in the maxilla under the sinus membrane, an irrigation fluid may be introduced between the flat surface of the tip and the sinus membrane which is then raised by cavitation of the irrigation fluid. When ultrasound vibrations are transmitted to the tip, the irrigation fluid enters into cavitation immediately under the sinus membrane. The cavitation effect (micro-pressure oscillations) causes the gentle detachment and elevation of the membrane. Consequently, the ultrasonic tip of the invention may be used to lift the sinus membrane without coming in direct contact with it, further reducing the risks of tearing the membrane. The flat shape of the end of the ring-shaped part makes it possible to optimize the efficacy of the effect of ultrasound on the irrigation fluid.

In addition, the tip has a bent portion at this central part, namely between the proximal part and the distal part of the tip.

According to a special aspect of the present invention, the internal irrigation channel emerges in the center of the flat surface, in order to induce homogeneous cavitation of the irrigation fluid.

According to another aspect of the invention, the flat surface presents at its periphery a rounded edge which further minimizes the risks of tearing the membrane in the case of contact with this part of the tip.

The flat surface at the end of the annular section has a diameter which is preferably similar, i.e. slightly lower, than that of the drilled hole made in the bone in order to allow the insertion of the aforesaid ring-shaped part. The flat surface may, for example, have a diameter of approximately 3 mm.

Another object of the invention is an ultrasonic dental surgery device consisting of at least one surgical handpiece connected to an ultrasound generator containing means to feed the handpiece with irrigation fluid. The aforesaid handpiece comprising the means to generate ultrasound vibrations and the means to distribute irrigation fluid delivered by the ultrasound generator, is characterized in that it comprises at least one ultrasonic tip as described above, the aforementioned tip being mechanically coupled to the handpiece ultrasound generator and the irrigation channel of the tip also being in communication with the means of distributing irrigation fluid of the aforesaid handpiece.

The invention also relates to a method for lifting a sinus membrane located between a sinus and a maxilla, said method including the drilling of a hole in the maxilla reaching the sinus membrane, wherein said method further comprises the steps of introducing in said hole an ultrasonic tip and setting up ultrasound vibrations to the tip while feeding the inner irrigation channel of said tip with irrigation fluid.

### BRIEF DESCRIPTION OF DRAWINGS

Other characteristics and advantages of the invention are demonstrated by the following description of particular ways of devising the invention which are given as nonrestrictive examples, in reference to the appended drawings, on which:

FIGS. 1A with 1D, described above, show a bone augmentation operation in a maxilla according to a previous procedure,

FIGS. 2 and 3 show cross-sections and a perspective drawing of the ultrasound tip according to an embodiment of the invention,

FIG. 4 shows an ultrasonic dental surgery device fitted out with the tip of FIGS. 2 and 3;

FIG. 5 is a cross-sectional view of the handpiece of FIG. 4 and

FIGS. 6A to 6D, show a bone augmentation operation in a maxilla in which the elevation of the sinus membrane is achieved using the tip described in this invention.

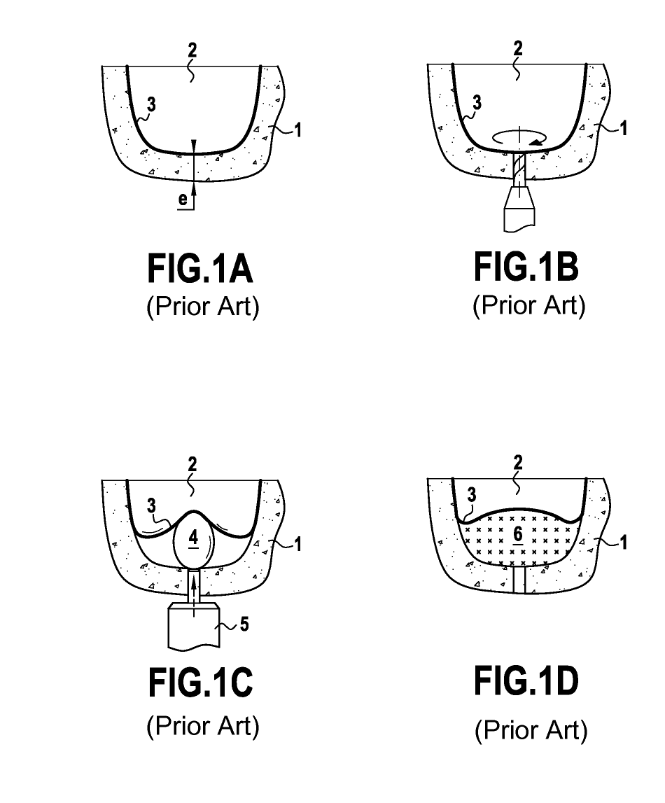

### DETAILED DESCRIPTION OF EMBODIMENTS OF THE INVENTION

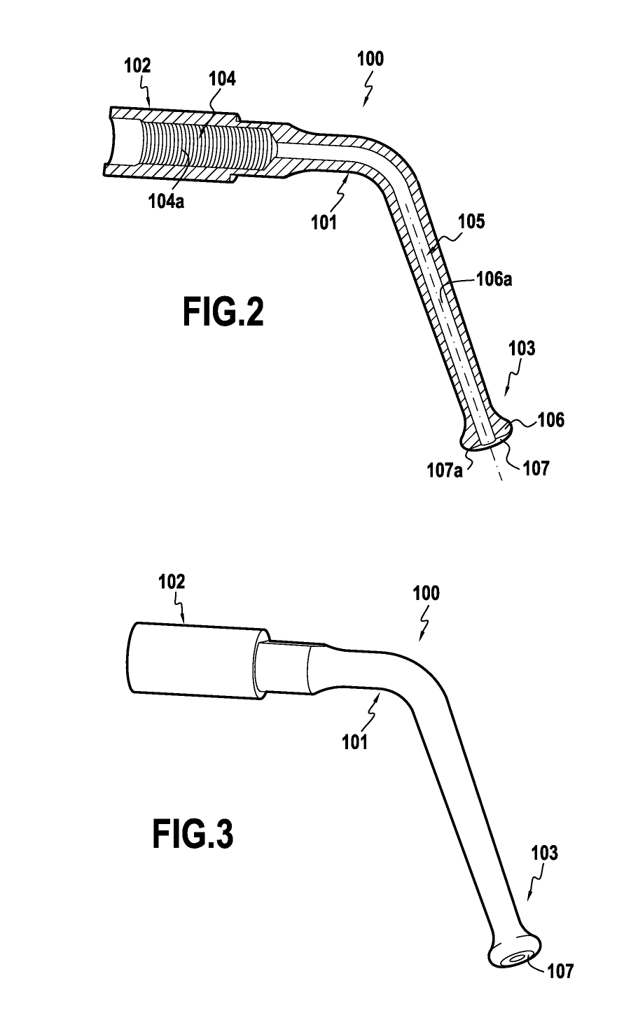

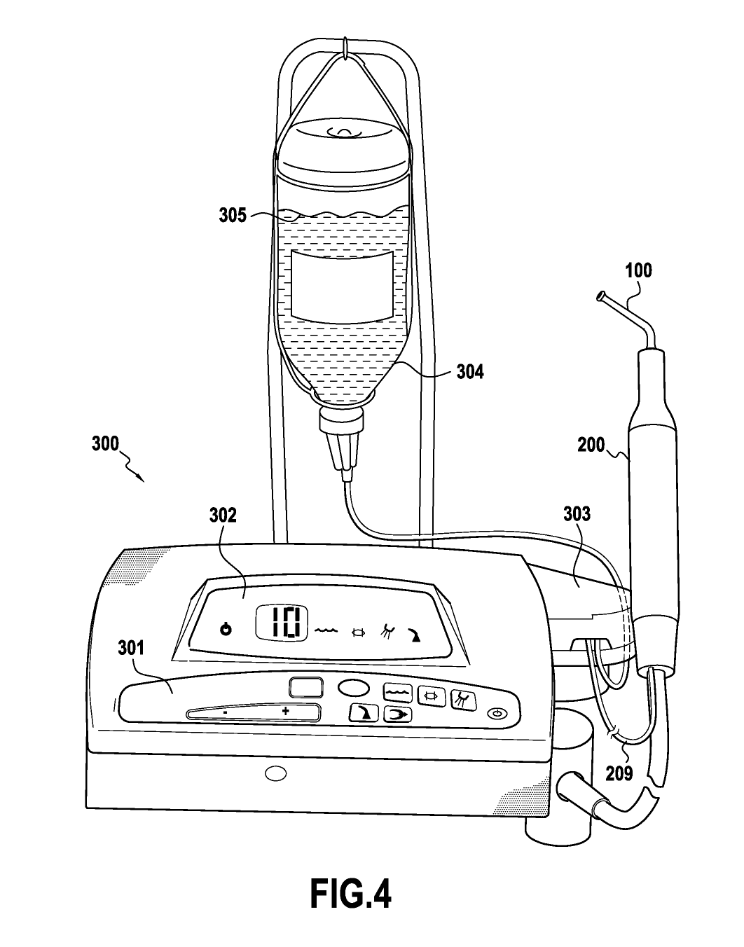

FIGS. 2 and 3 show an ultrasound tip 100 used to lift the sinus membrane according to an embodiment of the present invention.

Ultrasound tip 100 is formed of a body 101, for example in a metallic material, extending between a proximal part 102 intended to be mechanically coupled to a surgical handpiece generating vibrations as explained below and a distal part 103 intended to reproduce the vibrations transmitted by the handpiece. The proximal part 102, extending along a longitudinal axis (101a), comprises a cavity 104, the walls of which are threaded 104a to allow the tip to be attached to the handpiece. The cavity 104 continues in an internal irrigation channel 105 which extends inside body 101 for all its length and emerges at the end of the distal part 103.

The distal part 103 of tip 100 extends along a longitudinal axis 101b. The distal parts 103 corresponds to the part of the tip which is introduced into the mouth of the patient for reaching the hole drilled in the bone in order to carry out the elevation of the sinus membrane. In the embodiment presented in FIGS. 2 and 3, the distal part 103 comprises a ring-shaped part 106 revolving around the axis 101b which, at this part the tip, consists in the axis of the internal irrigation channel 105. The ring-shaped part 106 has a cross-section which increases towards the free end of distal part 103. The end of the ring-shaped part 106 of increasing cross section has a flat area 107 which corresponds to the part of the tip which will be placed facing the sinus membrane to be lifted. The flat surface 107 preferably has at its periphery a rounded edge 107a rather than a projecting edge, in order to reduce the risks of damaging the sinus membrane if the tip comes in contact with the membrane.

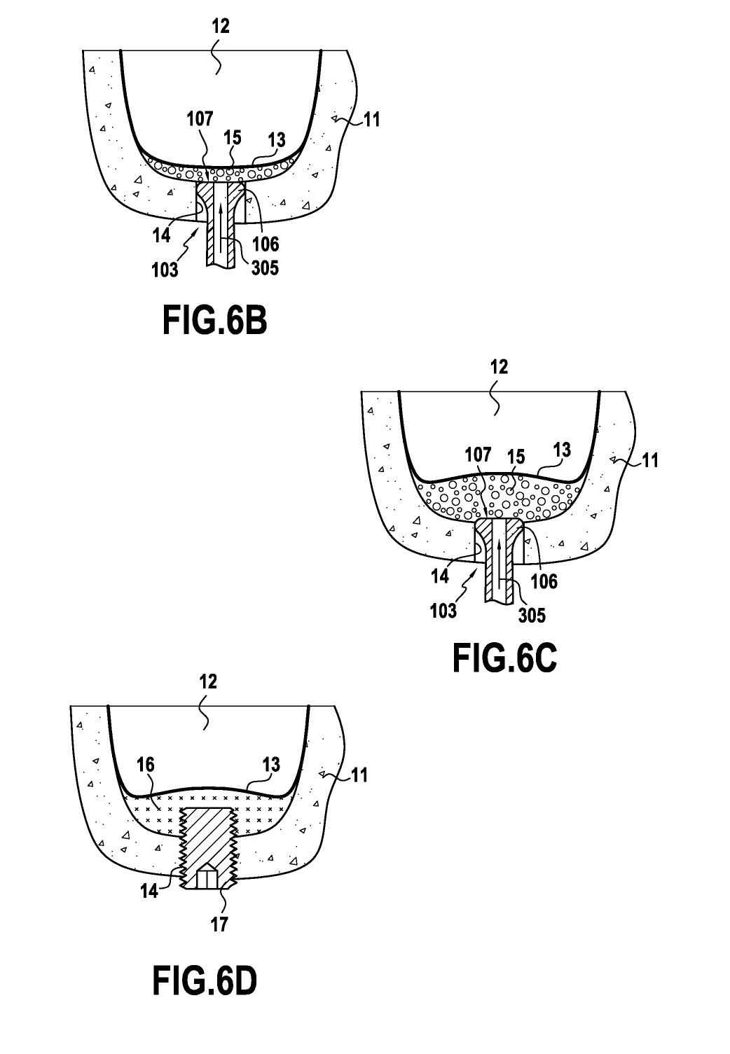

As shown in FIG. 2, the body 100 has a bent portion 108 between the proximal part 102 and the distal part 103. More precisely, the longitudinal axis 101b of the distal part 13 forms with a longitudinal axis 101a of the proximal part 102 a bend angle. With such an inclination of the distal part, the physician may introduce the free end of the tip in the hole drilled in the maxilla from the mouth of the patient while ensuring the maintaining of the flat surface in a parallel position to the sinus membrane as represented in FIGS. 6A and 6B which will be described later.

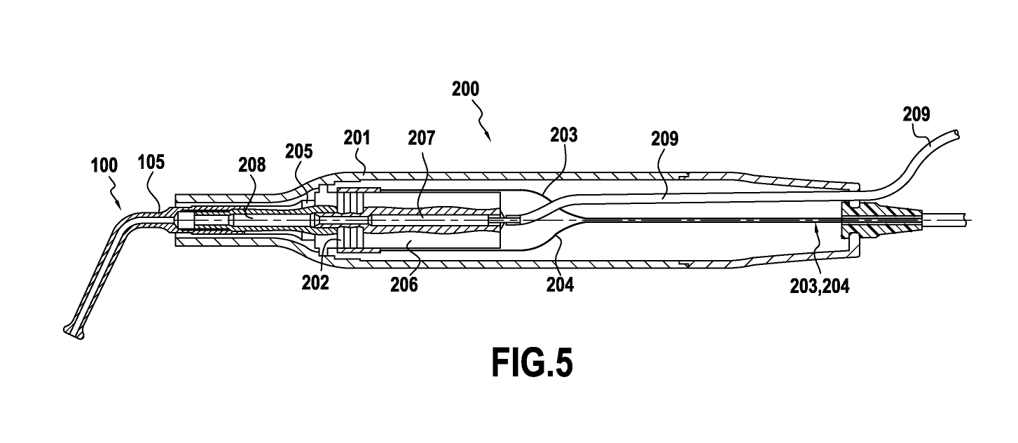

FIG. 4 shows an ultrasound treatment device comprising an ultrasound generator 300 connected to a handpiece 200 equipped with ultrasonic tip 100 described above.

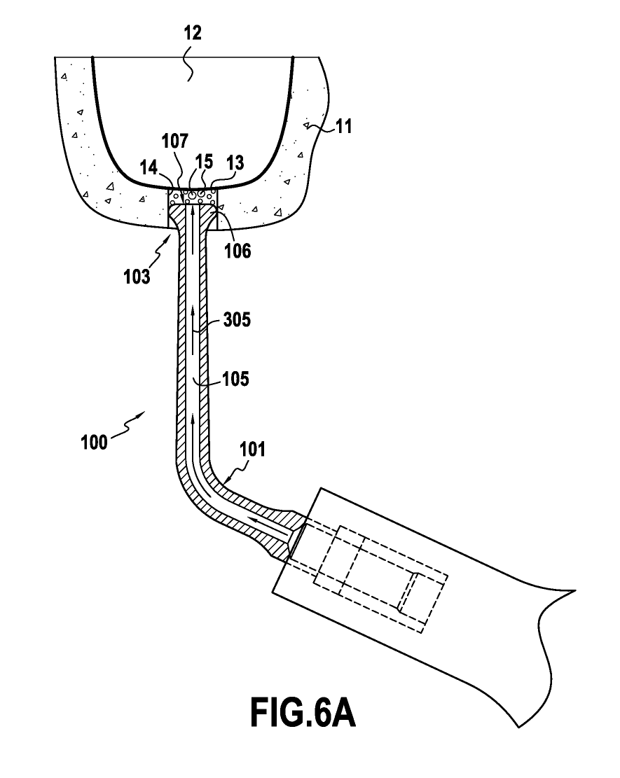

As shown on FIG. 5, handpiece 200 comprises a cylindrical hollow body 201 made from an insulating material which contains, a transducer 202, composed of a stack of piezoelectric chips for example, electrically connected to the ultrasound generator of 300 by electric power conductors 203, 204.

A vibration amplifier 205 is in contact with the anterior face of transducer 202. Tip 100 is fixed, for example by screwing, to the end of amplifier 205 so as to be mechanically coupled with transducer 202. Tip 100 is then subjected to a longitudinal vibratory movement when transducer 202 is supplied with high frequency alternating current controlled by generator 300. The power and amplitude of the ultrasound waves transmitted to the tip are controlled from generator 300 by controls 301 (buttons) and a display system 302 of selected controls.

Transducer 202 is in contact with a counterweight 206 in the center of which is a channel 207 communicating on one side with channel 208 created in the amplifier 205 and, on the other with a flexible tubing 209 connected to a pump 303 of ultrasound generator 300. The ultrasound generator 300 also contains a source 304 of an irrigation fluid which is connected to pump 303. Consequently, when pump 303 is activated (using the control unit 301) irrigation fluid 305 from source 304 is driven in turn into tube 209, channel 207 of the counterweight and channel 208 of the amplifier 205 which communicates with the inner irrigation channel of tip 100.

The operation of such an ultrasound treatment system is well documented and will not be described in more detail here.

We describe below with reference to FIGS. 6A with 6D, a sinus-lift procedure performed using the ultrasound tip and the ultrasound treatment device described above.

FIG. 6A shows the start of the elevation of the sinus membrane 13 separating a sinus 12 from a maxilla 11. The ring-shaped part 106 with increasing cross section of the distal part of tip 103 is inserted into the hole 14 previously drilled in the maxilla 11 in order to reach the sinus membrane 13. Once the ring-shaped part 106 is introduced into the hole 14, the physician actuates the ultrasound generator both to set up ultrasound vibrations in the tip and to feed the site with irrigation fluid via the inner irrigation channel 105 of the tip. The flat surface 107 in the center of which emerges the irrigation channel, makes it possible when ultrasound vibrations are transmitted to the tip and an irrigation fluid emerges from channel 105, to create a cavitation effect immediately under the sinus membrane and to lift it without contact with the tip. Irrigation fluid 305 emerging from the outlet of channel 105, lies between the sinus membrane 13 and the flat surface 107, which is driven by an ultrasonic vibratory movement. The action of ultrasound in liquid media is well documented and involves a cavitation phenomenon leading to the creation, growth and implosion of bubbles formed when a liquid is subjected to a periodic pressure wave. Under the effect of the ultrasound vibrations of the flat surface 107, hydrodynamic cavitation bubbles 15 (micro-pressures) are formed in irrigation fluid 305 which implode (negative pressure) in contact with the solid surfaces that they encounter and in particular the sinus membrane 13. These pressure oscillations create a pneumatic effect on the membrane leading to the gentle and gradual elevation of the membrane.

Because of the presence of flat surface 107, there is little risk of tearing the sinus membrane if it comes in contact with the tip contrary to the case for instruments that have a blunt or similar shape.

Moreover, the elevation of the membrane was shown to be obtained by cavitation of irrigation fluid 305 located between the tip and the membrane, i.e. without contact between the sinus membrane 13 and tip 100, which further reduces the risks of tearing the membrane by the tip.

FIGS. 6B and 6C show the progressive elevation of sinus membrane 13 obtained in particular by slightly advancing the flat surface 107 inside sinus 12 while keeping the flat surface generally parallel with the sinus membrane 13.

The use of an irrigation fluid makes it possible to obtain a relatively uniform elevation over the whole width of the membrane. Indeed, as illustrated by FIG. 6B, the irrigation fluid 305 introduced under membrane 13 spreads throughout the floor of sinus 12. The irrigation fluid then exerts a more or less uniform lifting force over a wide surface area of the membrane, allowing a balanced elevation of the membrane. This is not the case for instruments with only a single point of contact on a small part of the membrane. Moreover, in order to obtain a uniform lift of the sinus membrane during at least the first moments of the elevation, the flat surface 107 of the tip 100 should be introduced and maintained in a position approximately parallel to the membrane as represented in FIGS. 6A and 6B. The bent portion 108 of the tip 100 facilitates such a positioning because the tip can be oriented or positioned by the physician without being disturbed by the lower jaw of the patient.

When a sufficient volume has been freed under the membrane, this volume is filled by a bone graft 16 allowing the placement of an implant 17 as shown on FIG. 6D.

The body of ultrasound tip 100 described above has an angled shape facilitating the insertion of the distal part in the hole drilled in the maxilla inside the patient's mouth. The angle of the curve formed between the proximal and distal parts may vary depending on the site to be reached. The body of the tip according to the invention may have other shapes to enable access to specific sites.

The shape of the ring-shaped part formed at the level of the distal part of the tip may have different profiles. The ultrasound tip described above has a ring-shaped part of increasing cross section which presents a curved profile (nonlinear increase in the cross-sectional area of the ring-shaped part). The distal part of the tip according to the invention may also have a cylindrical part which widens according to a rectilinear profile (linear increase in the cross-sectional area of the cylindrical portion) or other shape.

What is claimed:

1. Method for lifting a sinus membrane located between a sinus and a maxilla, said method including the drilling of a hole of a given diameter in the maxilla reaching the sinus membrane, wherein said method further comprises the steps:
introducing in said hole an ultrasonic tip having a distal part including a ring-shaped part of increasing cross-section with a distal end formed by a flat area extending generally perpendicular to the longitudinal axis of said distal part and wherein said flat area has a distal side facing away from said proximal part and is bordered by a rounding, said ring shaped part being larger than the body of the tip but closely corresponding to the diameter of the hole, and said tip including an internal irrigation channel emerging on a distal side of said flat area; and
while the tip is within the hole, advancing the tip towards the membrane while setting up ultrasound vibrations to the tip and feeding the internal irrigation channel of said tip with irrigation fluid to thereby create a cavitation effect in irrigation fluid between the distal part and the membrane and a lifting force for gently detaching and elevating the sinus membrane without direct contact between the membrane and the distal part of the tip.

2. The method according to claim 1, including using as a body of the tip a body that has a bend between said proximal part and said distal part, and during the step of introducing the ultrasonic tip in said hole and setting up ultrasonic vibrations to the tip, maintaining the flat area of the end of the distal area generally parallel with the sinus membrane.
